(19) United States
(12) Patent Application Publication
(10) Pub. No.: US 2020/0171317 A1
(43) Pub. Date: Jun. 4, 2020

(54) System for its use in a tumor treatment

(71) Applicant: Mohamed A Basiony, Kenmore, WA (US)

(72) Inventor: Mohamed A Basiony, Kenmore, WA (US)

(21) Appl. No.: 16/699,711

(22) Filed: Dec. 1, 2019

Related U.S. Application Data
(60) Provisional application No. 62/774,283, filed on Dec. 2, 2018.

(51) Int. Cl. A61N 1/44; A61B 17/34; A61N 1/02

(52) U.S. Cl. CPC A61N 1/44; A61N 1/025; A61B 17/3468

(57) ABSTRACT

A system to be used in a tumor treatment, comprises a trocar, a device and connection cables. A trocar that is used to puncture a patient's body at entry site to a tumor location, comprises a first portion with a cutting edge and a heat generating element, an elongated body and a second portion. A device that is connected to a trocar to provide a heat generating element of a trocar with a necessary energy. A device has a radiofrequency current generator to supply and control the energy supplied to a heat generating element.

In operation, there are two steps, in a first step, a trocar is inserted into a tumor location, then a salty solution is pump within a tumor area via a syringe and oblique ports of a heat generating element to cause tumor's cells to shrink. Then after a shrink effect a salty solution is sucked via a syringe. In a second step of operation, a higher temperature may be achieved in cancer cells via the activation of a heat generating element to produce a precise and adequate temperature intracellularly to kill cancer cells.

### BACKGROUND

Hyperthermia has been recognized to interact synergistically with ionizing radiation and chemotherapy treatments, a factor which augments its clinical utility as an anticancer modality.

In hyperthermia treatment of a tumor, localized particles that are magnetically-coupled or light-coupled to an external electromagnetic energy or light energy may be used to generate a heat in an area of a patient's tissue that is containing both normal and malignant cells to raise a temperature of such area to a range of 41-44 C to may destroy of malignant cells.

It has been known that a physical characteristic that differentiates cancer cells from normal cells is that cancer cells die at a lower temperature than do normal cells. A temperature at which a normal cell will be killed and thereby irreversibly will not be able to preform its normal cell functions may be a temperature of 46.5 C, on average. The cancer cells in contrast may be killed at a lower temperature of 45.5 C. Therefore, with a given precisely controlled temperature elevation in an area of a patient's tissue that is containing both normal and malignant cells may lead to selectively destroy cancer cells before the death of normal cells.

To achieve these higher temperatures in cancer cells as in induced hyperthermia, an external electromagnetic energy or light energy capable of generation of heat in localized particles may be used to induce selective thermal death of cancer cells. While, in direct hyperthermia, a direct implementation of a heating device may be used in the area of cancer cells.

The major obstacles impeding the wide spread clinically utilization of hyperthermia in treating carcinomata may be: no precise heat control, no precise control on the number of particles that are accumulated inside cancer cells to achieve such heat effect, intravenously injection of the particles, a long time necessary to inductively heat the particles and the accumulation of particles at the kidney and liver of a patient which may create serious side effects after treatment.

While the obstacle, in direct hyperthermia, may be it was treating cancer extracellularly as an outer membrane of a cancer cell that is composed of lipids and proteins may be a poor thermal conductor thus may making it difficult for an application of a direct heat by means to penetrate to an interior of a cell where the intracellular temperature must be raised to effect the death of a cell. In this prior technique, a temperature may be raised so high to affect an adequate intercellular temperature to kill cancer cells. This temperature raise may lead to destroy normal cells adjacent to the application of the heat.

Therefore, there is a need to enhance a hyperthermia therapy to may address the above obstacles.

### SUMMARY

Accordingly, a system comprises a trocar, a device and connection cables is described to may address the above issues.

A trocar is used to puncture a patient's body at entry site to a tumor location and may include a first portion with a cutting edge, an elongated body and a second portion. A syringe is connected to a trocar's second portion via a coupling assembly.

A device that is connected to a trocar to provide a heat generating element of a trocar's first portion with a necessary energy. A device may have a radiofrequency current generator to supply and control the energy supplied to a heat generating element. Also, a device may have an OFF/ON switch, a touch screen, a menu key, a confirm key, a start key and a reset key to set up; a temperature of a heat generating element, an operation time, a diameter of a heat generating element and a diameter of a tumor. Based on such operational data, a device may be able to produce and control precise energy dissipation to produce a rapid rise in temperature inside a tumor's tissue. A device also, may display an actual temperature measured by a temperature sensor and an actual radiofrequency energy being used.

In operation, there are two steps after the above pre-set step, in a first step, a trocar is inserted into a tumor location, then a salty solution is pump within a tumor area via a syringe and oblique ports of a heat generating element (that is not yet heated) to may cause tumor's cells to shrink due to water movement across cells' membranes toward a higher concentration of salt that is outside of the cells membranes to bond with it (osmosis process). Further, excess salt may knock the peripheral membranes proteins off cells and may unfold proteins, rendering them non-functional to may allow for hyperthermally treatment of cancer cells intracellularly. Then after a shrink effect and proteins unfold, a salty solution is sucked via a syringe.

In a second step of operation, a higher temperature may be achieved in cancer cells via the activation of a heat generating element to produce a precise and adequate temperature intracellularly to kill cancer cells.

### BRIEF DESCRIPTION OF THE DRAWINGS

The accompanying drawings, which are incorporated in, and constitute a part of the specification, illustrate or exemplify embodiment of the present implementation and, together with the description, generally explain the principles and features of the present implementation. The drawings are briefly described as follows:

FIG. 1 illustrates a perspective view of the system with its components according to the present disclosure.

FIG. 2 is an enlarged perspective view of the trocar of the system in FIG. 1 according to the present disclosure.

FIG. 2a is a cross-sectional view of the elongated body of the trocar of the system of FIG. 1 taken along the lines 2a-2a according to the present disclosure.

FIG. 3 is an enlarged perspective view of the device of the system of FIG. 1 according to the present disclosure.

FIG. 4 is a schematic view of the trocar of FIG. 1 in step one of operation according to the present disclosure.

FIG. 5 is a schematic view of the trocar of FIG. 1 in step two of operation according to the present disclosure.

### DETAILED DESCRIPTION

The following detailed description illustrates the principal of the disclosure by way of example not by way of limitation. While a reference use of the present disclosure describes the system to be used in a tumor treatment, it will be understood that the system may also be used for other types of treatments, consequently, the scope of the implementation is not to be limited by the field to which the implementation is applied.

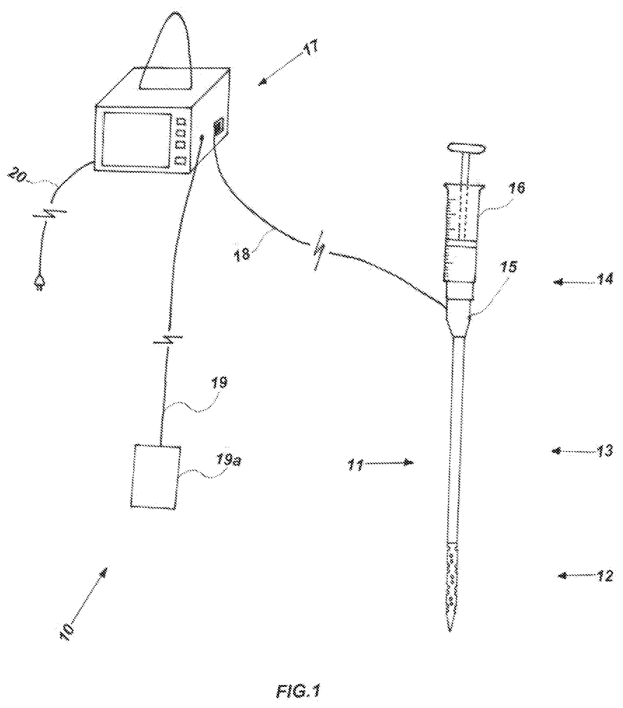

FIG. 1 illustrates a system 10 comprises a trocar 11, a device 17, a connection cable 18 to connect a trocar 11 to a device 17 and a connection cable 19 to connect a patient ground plate 19a to a device 17. A trocar 11 is used to puncture a patient's body at entry site to a tumor location and may include a first portion 12 with a cutting edge, an elongated body 13 and a second portion 14. A syringe 16 of a second portion 14 is connected to an elongated body 13 via a coupling assembly 15. A device 17 may receive its power via a cable 20.

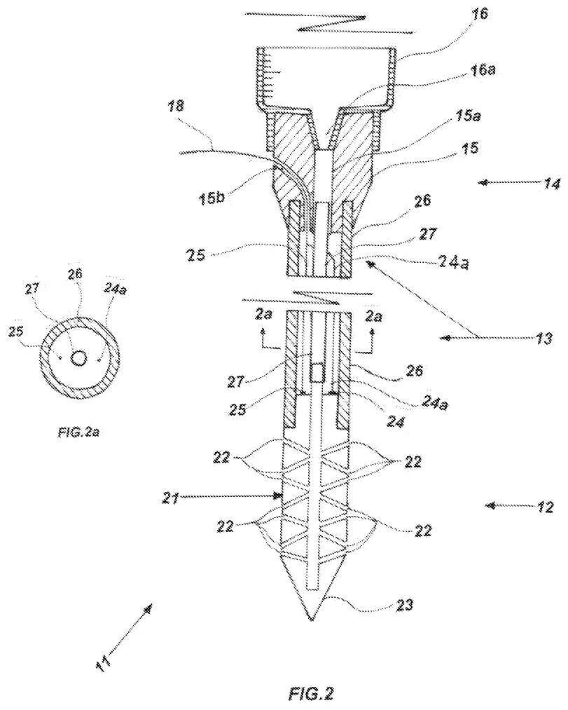

FIG. 2 illustrates an enlarged perspective view of a trocar 11 of a system 10 that comprises a first portion 12, an elongated body 13 and a second portion 14. A first portion 12 of a trocar 11 comprises a heat generating element 21, oblique ports 22 and a cutting edge 23. An elongated body 13 that connects a first portion 12 to a second portion 14 comprises an outer tube 26 and an inner tube 27. A second portion 14 comprises a coupling assembly 15 for sealingly coupled a second portion 14 to a syringe 16 and to an elongated body 13. A coupling assembly 15 may have an inner channel 15a to fluidly connect a syringe tip 16a to an inner tube 27 and oblique ports 22 of a heat generating element 21 and also may have an inner channel 15b for a single radiofrequency lead 25 that power a heat generating element 21 and temperature sensor leads 24a of a temperature sensor 24. These leads may be connected to a device 17 via a cable 18.

A heat generating element 21 may be made of a metal such as surgical stainless steel or any other suitable materials which may be sufficiently hard to hold a cutting edge 23 and also rigid to withstand twisting through a patient's tissue. Also, a heat generating element 21 may have a unit heat capacity of about less than one joule/degree C. and an exterior surface that may be coated with a non-steak material to do not stick to a tumor tissue.

Also, a heat generating element 21 may have a sufficient mass to avoid burn-through during use and to provide a sufficient heat to rise a temperature in cancer cells to a range of 41 to 44 C for a preferential destruction of malignant cells. Normal tissue may be destroyed at a temperature of about 46.5 C. A diameter of a heat generation element 21 may be in a range from 8 Fr to 20 Fr or any suitable diameter based on the size of a tumor.

Outer tube 26 and inner tube 27 may be formed of a polycarbonate material or a medical grade PVC or any suitable material with different hardness based on the location of a tumor. In one implementation, a coupling assembly 15 may also be formed of the same material as outer tube 26 and inner tube 27. In another implementation, a coupling assembly 15 may be formed of a different material.

An outer tube 26 may have the same diameter as a heat generating element 21. In one implementation a trocar 11 may be have a straight extended length. In another implementation, a trocar 11 may have a bend that is preferably formed between a first portion 12 and an elongated body 13. The angle of bend may be any suitable angle which facilitates a movement, and guidance of a trocar 11 through a patient's tissue. A trocar may have different lengths to be used with different tumor's locations.

A cutting edge 23 of a heat generating element 21 of a first portion 12 is formed at a distal end of a heat generating element 21 and may be produced by a milling process or any suitable process with a sharper end for an easy and smooth insertion. Also, a cutting edge 23 may be coated by a radiopaque material for a fluoroscopic observation.

FIG. 2a illustrates a cross-sectional view of an elongated body 13 taken along the line 2a-2a, wherein an outer tube 26, an inner tube 27, a single radiofrequency lead 25 to may energize a heat generating element 21 and temperature sensor leads 24a of a temperature sensor 24 which may be used to measure an actual temperature of a heat generating element 21.

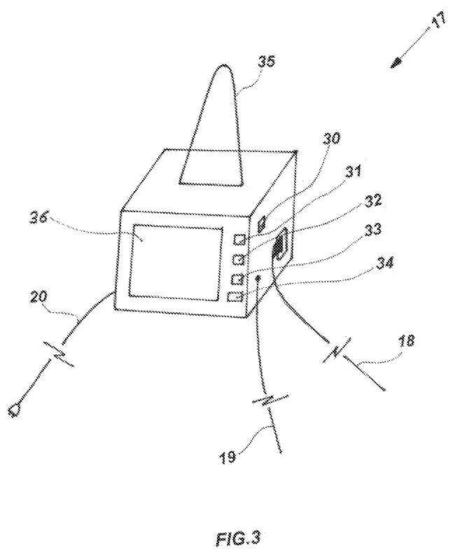

FIG. 3 illustrates an enlarged view of a device 17 which is used to supply and control an energy supplied to a heat generating element 21 of a first portion 12 of a trocar 11. A device 17 may have a radiofrequency current generator (not shown for simplicity) to supply a heating generating element 21 with a necessary radiofrequency continuous or pulsed energy. Radiofrequency energy may be safer compare to a direct current or a low frequency power sources as the risk of a physiological response or electrocution response may be reduced at a radiofrequency above 100 Khz. The dissipated electrical energy is converted into heat to produce an adequate heating to the area of a patient's tissue containing malignant cells to raise the temperature of such area to the range of 41 to 44 C.

A device 17 may have an OFF/ON switch 30 for power activation, a touch screen 36, a menu key 31, a confirm key 32, a start key 33, a reset key 34 (to reset a device 17 after an alarm), a cable 18 to connect a single radiofrequency lead 25 and temperature sensor leads 24a to a device 17, a cable 19 to connect a patient ground plate 19a to a device 17, a belt 35 to hang a device 17 on IV Pole and a power cord 20 to power a device 17.

Before the operation, an operator presses a menu key 31 to choose the operational parameters such as; Temperature, Time, Diameter—heat generating element and Diameter—tumor area, then using a touch screen 36 to select a parameter after parameter for set-up. For Temperature, an operator may use a soft numeric key pad on a touch screen 36 to set-up the required temperature, for example 43 C then press confirm key 32 for confirmation.

For time, an operator may set an operational time in a range from 5-60 seconds or any suitable duration based on the type of tumor and its size, using a soft numeric key on a touch screen 36 and then press confirm key 32 for confirmation. A heating energy is activated for a such preset time to avoid deep heat penetration and hence thermal necrosis to surrounding normal tissues.

For Diameter—heat generating element, an operator can set an actual diameter using a soft numeric key on a touch screen 36, for example 12 Fr and press confirm key 32 for confirmation. For Diameter—tumor area, based on approximately an actual tumor area, for example 30 mm, an operator may set it using a soft numeric key on touch screen 36, then press confirm key 32 for confirmation. Now, a device 17 is ready for operation.

Based on an operational data, a device 17 may produce and control a precise temperature and an energy dissipation to produce a rapid rise in temperature inside a tumor's tissue based on a Fourier equation (1).

$$Q = \frac{KA ( T 1 - T 2 )}{B} \qquad ( 1 )$$

Wherein; Q is a heat (thermal energy) that is transferred from a heat generating element 21 to a tumor tissue and is proportional to a cross-sectional area A of a tumor tissue; Temperature T1 is a set-up temperature (for example 43 C) and T2 is a temperature of a tumor tissue before operation (which may be approximately 36.6 C); and B is a tumor thickness (which is approximately equals to (radius of a tumor—radius of a heat generating element)); and K is a thermal conductivity of a tumor (constant).

The device also, may display an actual temperature that may be measured by a temperature sensor 24, an actual radiofrequency energy being used, an operation status, and alarm status on a touch screen 36.

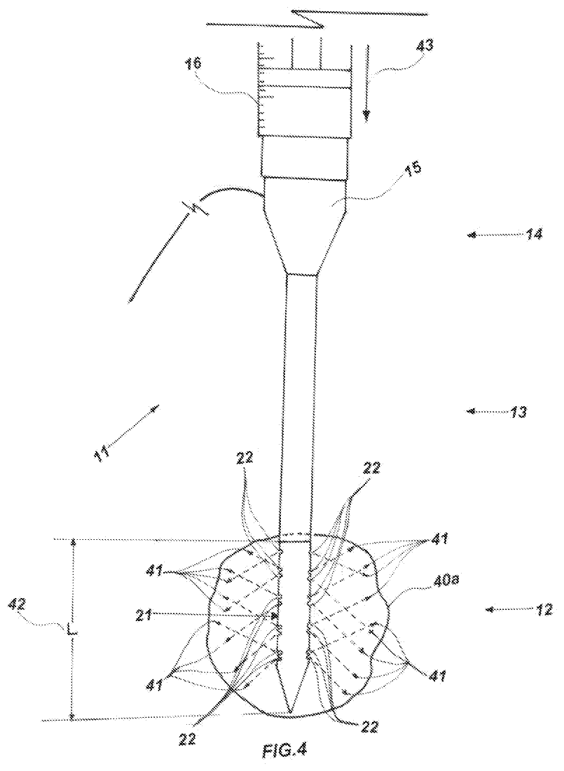

In operation, there are two steps after a pre-set step, in a first step as in FIG. 4, a trocar 11 that comprises of a first portion 12, an elongated body 13 and second portion 14, may be inserted into a tumor area 40a, then an salty solution 41 may be pumped as per arrow 43 within a tumor area via a syringe 16 through a coupling assembly 15, inner tube 27 (not shown for simplicity) and oblique ports 22 of a heat generating element 21 of a first portion 12 to may cause tumor's cells to shrink due to a water movement across cells' membranes toward a higher concentration of salt that is outside of the cells membranes to bond with it (osmosis process).

Further, excess salt may knock the peripheral membranes proteins off cells and may unfold proteins, rendering them non-functional to may allow for hyperthermally treatment of cancer cells intracellularly. Peripheral proteins can be dissociated from the membrane by treatment with salt solutions or by changes in pH (treatments that disrupt hydrogen bonds and ionic interactions) (Dr. Reginald Garrett and Dr. Charles Grisham book “Biochemistry”; 246, fourth edition). Then after that an extracellular salty solution may be sucked via a syringe 16.

Salty solution such as a sodium chloride (NaCl) may be used or any other suitable solutions. A solution with a salinity of range of about 3% to about 20% may be used or any other suitable range. A preferable concentration of a salty solution may be of 4%-6%. In another implementation, a concentration of 20% may be used to kill bacteria in case of bacteria induced cancer.

Also, FIG. 4 shows a length “L” 42 which is the length of a heat generating element 21 that is approximately equal to a tumor diameter, also to avoid deep heat penetration and hence thermal necrosis to surrounding normal tissues. This may mean that a trocar may have different lengths of a heat generating element 21 based on a tumor size.

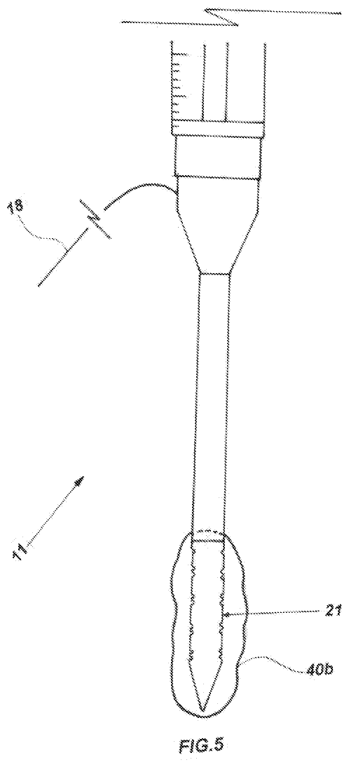

In a second step of operation as in FIG. 5 which may be related to apply a radiofrequency energy to a tumor area 40b (after it has been shrined due to a salty solution as in step 1 of operation). To activate a heating process, an operator must press a start key 33 on a device 17 to activate a device 17 to may produce a precise presetting higher temperature in a tumor area 40b through an activation of a heat generating element 21 via a cable 18 to kill cancer cells. Due to a first step of operation (using an acidic solution) cancer cells may be killed by a higher intracellularly temperature as described above.

1. A system comprising a trocar, a device and connection cables.
Said trocar comprises a first portion with a cutting edge, an elongated body and a second portion;
A first portion of said trocar comprises a heat generating element, oblique ports and a cutting edge;
An elongated body of said trocar that connects said first portion to said second portion comprises an outer tube and an inner tube;
A second portion of said trocar comprises a coupling assembly for sealingly coupled a second portion to a syringe and to an elongated body. Said coupling assembly has an inner channel to fluidly connect a syringe tip to said inner tube and said oblique ports of said heat generating element and has an inner channel for a single radiofrequency lead that power said heat generating element and temperature sensor leads of a temperature sensor;
A device has a radiofrequency current generator to supply said heating generating element with the necessary radiofrequency continuous or pulsed energy. Said device has an OFF/ON, a touch screen, a menu key, a confirm key, a start key, a reset key 34, a cable to connect said single radiofrequency lead and temperature sensor leads to said device, a cable to connect a patient ground plate to said device, a belt and a power cord.

2. The system of claim 1, wherein said trocar has different straight or bend configurations.

3. The system of claim 1, wherein said trocar has different lengths and diameters.

4. The system o claim 1, wherein said bend configuration is formed between said first portion and said elongated body.

5. The system of claim 1, wherein said heat generating element of said first portion is made of sufficient hard and rigid metal material.

6. The system of claim 1, wherein said heat generating element is coated with a non-steak material.

7. The system of claim 1, wherein the length of said heat generating element is approximately equal to the tumor diameter.

8. The system of claim 1, wherein said cutting edge of said heat generating element is formed at the distal end of said heat generating element with sharper end.

9. The system of claim 1, wherein said cutting edge is coated by a radiopaque material.

10. The system of claim 1, wherein said heat generating element contacts with said temperature sensor and said single radiofrequency lead.

11. The system of claim 1, wherein said temperature sensor is used to measure the actual temperature that is displayed on said device's screen.

12. The system of claim 1, wherein said single radiofrequency lead is used to supply said heat generating element with necessary continuous or pulsed radiofrequency energy.

13. The system of claim 1, wherein said inner and outer tubes are formed of different hardness plastic materials.

14. A process comprising:
Connecting system's components together;
Setting the operational parameters and locating said patient ground plate at its suitable place;
Inserting said trocar into a tumor area;
Using associated syringe to pump a salty solution into a tumor area.
Sucking the salty solution after a tumor has been shrinked and the proteins of peripheral membranes have been unfolded;
Activating a heating process of said heat generating element for certain preset time to kill cancer cells; and
Removing the trocar after treatment.

15. An operational process, comprising a preset-up step and two steps of operation:
In a preset-up step, the operator connects the system components together and place patient ground plate in a suitable place, then activating the device by pressing the ON/OFF key. Then presses a menu key to choose the operational parameters such as; Temperature, Time, Diameter—heat generating element and Diameter—tumor area, then using a touch screen to select a parameter after parameter for set-up. For Temperature, the operator may use a soft numeric key pad on a touch screen to set-up the required temperature, then press confirm key for confirmation. For time, an operator may set an operational time, using a soft numeric key on a touch screen and then press confirm key for confirmation. For Diameter—heat generating element, the operator sets an actual diameter using a soft numeric key on a touch screen and press confirm key for confirmation. For Diameter—tumor area, the operator sets it using a soft numeric key on touch screen, then press confirm key for confirmation. Now, said system is ready for operation, by inserting said trocar into a tumor area,
In a step one of operation, a salty solution is pumped within a tumor area using the associated syringe through a coupling assembly, inner tube and oblique ports of a heat generating element to cause tumor's cells to shrink. Then after that an extracellular salty solution is sucked via said syringe.
In a step two of operation, the operator activates a heating process, by pressing a start key on said device to produce a precise presetting higher temperature in a tumor area through the activation of said heat generating element to kill cancer cells. Then, the operator removes the trocar.

16. The process of claim 15, wherein the length of said heat generating element is approximately equals to the tumor diameter.
